(19) United States
(12) Patent Application Publication
(10) Pub. No.: US 2006/0237866 A1
(43) Pub. Date: Oct. 26, 2006

(54) Method and apparatus for preparing nanoparticles

(75) Inventors: Kyo-yeol Lee, Yongin-si (KR); Yoon-ho Khang, Yongin-si (KR)

Correspondence Address: BUCHANAN, INGERSOLL & ROONEY PC, POST OFFICE BOX 1404, ALEXANDRIA, VA, 22313-1404, US

(73) Assignee: SAMSUNG ELECTRONICS CO., LTD., Suwon-si (KR)

(21) Appl. No.: 11/268,482

(22) Filed: Nov. 8, 2005

(30) Foreign Application Priority Data
Nov. 24, 2004 (KR) 10-2004-0097008

(51) Int. Cl. H01L 21/00; B29B 9/00; A61K 9/14; C23C 16/00

(52) U.S. Cl. 264/5; 424/489; 977/906; 438/1; 118/723 R

(57) ABSTRACT

Provided are an apparatus and method for producing nanoparticles by gas-to-particle conversion. Products obtained by decomposing a precursor and initial aggregated products thereof have the same polarities due to gas discharge so that decomposition products or initial aggregated products thereof are aggregated to produce nanoparticles with a narrow size distribution.

### CROSS-REFERENCE TO RELATED PATENT APPLICATIONS

This application claims the benefit of Korean Patent Application No. 10-2004-0097008, filed on Nov. 24, 2004, in the Korean Intellectual Property Office, the disclosure of which is incorporated herein in its entirety by reference.

### BACKGROUND OF THE INVENTION

1. Field of the Invention

The present invention relates to a method and apparatus for preparing nanoparticles, and more particularly, to a method and apparatus for preparing nanoparticles with a narrow size distribution.

2. Description of the Related Art

Super-fine particles with a diameter of 1 nm to 100 nm are defined as nanoparticles which exhibit different properties from conventional bulk materials. In addition, properties of nanoparticles vary according to the sizes of the particles. Accordingly, a manufacturing technique for producing nanoparticles with a narrow size distribution around a desired size is required.

Conventional powder can be produced by breaking down a solid or liquid using, for example, mechanical grinding, spraying, or similar methods. That is, a bulk material is divided into small particles. However, the manufacture of nanoparticles starts from molecular units due to their small size.

Methods of preparing nanoparticles include gas phase synthesis methods and sol-gel processing methods. The gas phase synthesis methods include particle-to-particle conversion and gas-to-particle conversion. In the particle-to-particle conversion, a particle or liquid drop of a precursor that flows in a carrier gas is decreased in size using chemical reactions, thus producing a compound particle with a desired diameter. In the gas-to-particle conversion, a particle with the desired size is produced through chemical reactions with a precursor gas or by condensing a vaporized bulk material. That is, in the particle-to-particle conversion, the size of a particle that is sprayed is decreased to produce a nanoparticle. On the other hand, in the gas-to-particle conversion, the nanoparticle is produced by combining molecules.

As described above, since the properties of nanoparticles or nano-structure materials obtained using the nanoparticles are mainly determined by the size of the nanoparticles, a narrow size distribution of nanoparticles must be guaranteed to produce a material having uniform properties. Accordingly, the ability to obtain nanoparticles with a narrow size distribution is required for an apparatus for producing nanoparticles.

U.S. Pat. No. 6,586,785 discloses an apparatus for preparing nanoparticles by thermal decomposition chemical vapor deposition (CVD). However, when high-density nanoparticles are prepared using the apparatus, the distribution of sizes of nanoparticles is high because of low nucleation efficiency.

U.S. Pat. No. 6,230,572 by TSI instrument entitled ‘Instrument for Measuring and Classifying Nanometer Aerosols’ discloses an apparatus for classifying nanoparticles according to variations in electrical mobility resulting from different sizes. The apparatus can be used to measure the size distribution of pre-manufactured nanoparticles and to classify particles with a desired size. In other words, the apparatus is suitable for classifying pre-manufactured particles with a desired size, but not for producing nanoparticles with a narrow size distribution.

U.S. Pat. No. 5,075,257 discloses a method and apparatus for forming a silicon film. According to the method, a solid powder contains particles with an optimal diameter is electrically charged by corona discharge and the electrically charged particles are electrostatically arrayed at uniform intervals on a substrate, thus forming a high-quality film.

### SUMMARY OF THE INVENTION

The present invention provides a method of producing nanoparticles with a uniform size, that is, nanoparticles with a narrow size distribution.

The present invention also provides a method of producing high-density nanoparticles with a narrow size distribution.

The present invention also provides an apparatus for forming nanoparticles with a narrow size distribution by thermal decomposition.

The present invention also provides an apparatus for producing high-density nanoparticles with a narrow size distribution by thermal decomposition.

According to an aspect of the present invention, there is provided a method of producing nanoparticles using gas-to-particle conversion, wherein gas discharge is induced in products that are obtained by decomposing a precursor or initial aggregated products of the decomposed products so that the decomposed products of the precursor or the initial aggregated products thereof are electrically charged with the same polarities, thus forming nanoparticles with a narrow size distribution.

The gas-to-particle conversion may be performed by laser ablation, thermal decomposition, high-frequency sputtering, or plasma processing.

The method may further include: providing a precursor of a nanoparticle and a carrier gas to a thermal decomposition reactor to thermally decompose the precursor; electrically charging the thermally decomposed products of the precursor or the initial aggregated products thereof in the thermal decomposition reactor by corona discharge so that the thermally decomposed products of the precursor or the initial aggregated products thereof have the same polarities; and growing nanoparticles by allowing the thermally decomposed products or the initial aggregated products with the same polarities to be aggregated with neutral thermally decomposed products.

The operation of electrically charging may further include: providing a voltage that is lower than a voltage provided for corona discharge to a ring that contacts the thermal decomposition reactor and surrounds a portion where the corona discharge occurs, thus generating an electric field between the center of the corona discharge and the ring so that thermally decomposed products of the precursor or initial aggregated products thereof have the same polarities. In this case, an electric field may not be generated at an inner wall of the thermal decomposition reactor so that the density of produced nanoparticles can be increased.

The corona discharge may occur at a voltage of −4 kV to −20 kV, preferably, −7 kV to −10 kV.

The ring may be provided with a voltage of 0 kV to −10 kV, preferably, 0 to −4 kV.

The nanopaticle may be a nanoparticle of metal, an alloy, a ceramic, a semiconductor, or a composite compound.

According to another aspect of the present invention, there is provided an apparatus for producing nanoparticles, the apparatus including: a precursor inlet and a carrier gas inlet; a thermal decomposition reactor in which the mixture of the precursor and the carrier gas are introduced and the precursor is thermally decomposed and aggregated; and a corona discharge portion for corona discharge that is located in the thermal decomposition reactor and connected to a corona discharge voltage supplier.

The apparatus may further include a ring which contacts the thermal decomposition reactor and is connected to a ring voltage supplier and encompasses which the corona discharge portion, wherein the ring is supplied with a voltage that is lower than a voltage supplied for corona discharge to generate an electric field between the corona discharge portion and the ring. In this case, the electric field does not extend to the inner wall of the thermal decomposition reactor. By using the apparatus, high-density nanoparticles with a narrow size distribution can be manufactured.

The corona discharge may occur at a voltage of −4 kV to −20 kV, preferably, −7 kV to −10 kV.

The ring may be provided with a voltage of 0 kV to −10 kV, preferably, 0 to −4 kV.

The nanopaticle may be a nanoparticle of metal, an alloy, a ceramic, a semiconductor, or a composite compound.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of an apparatus for preparing nanoparticles by thermal decomposition according to an embodiment of the present invention;

FIG. 2A is a schematic view of an apparatus for preparing nanoparticles by thermal decomposition according to another embodiment of the present invention, wherein the apparatus of FIG. 2A is different from the apparatus of FIG. 1 in that a ring 6 is installed;

FIG. 2B is a vertical sectional view taken along line a-b shown in FIG. 2A;

FIG. 3 illustrates a direction in which an electric field is formed between a corona discharge portion 5 and a ring 6 of the apparatus shown in FIG. 2A;

FIG. 4 is a graph of sizes of nanoparticles and standard deviation of the sizes with respect to temperature for thermal decomposition according to Example 1 in which corona discharge occurred and Comparative Example 1 in which corona discharge did not occur;

FIG. 5 is a graph of density with respect to size of nanoparticles produced in a constant thermal decomposition reactor according to Example 2 in which corona discharge occurred and Comparative Example 2 in which corona discharge did not occur;

FIG. 6 is a graph of the size, density of nanoparticles, and the standard deviation of the sizes of the produced nanoparticles with respect to voltage provided for corona discharge when corona discharge occurred and a ring to which voltage was provided was installed in the thermal decomposition reactor; and

FIG. 7 is a graph of density with respect to size of nanoparticles according to Comparative Example 2 in which corona discharge did not occur, Example 2 in which corona discharge occurred, and Example 4 in which corona discharge occurred and a ring to which a voltage is applied was installed.

### DETAILED DESCRIPTION OF THE INVENTION

Hereinafter, a method and apparatus for preparing nanoparticles according to the present invention will be described with reference to a drawing that corresponds to an embodiment of the present invention.

A method of producing nanoparticles with a narrow size distribution according to an embodiment of the present invention is different from a conventional method of producing nanoparticles using gas-to-particle conversion in that a product obtained by decomposing a precursor or an initial aggregated product thereof is electrically charged by gas discharge.

The gas-to-particle conversion may be achieved by thermal decomposition, laser ablation, high-frequency sputtering, plasma processing, or the like, preferably, thermal decomposition. That is, the present method of producing nanoparticles can include any method in which gas particles grow into nanoparticles by aggregation.

The gas discharge which is performed to provide electric charges to the product obtained by decomposing a precursor or the initial aggregated product thereof may be obtained using plasma ion injection, corona discharge, or the like, preferably, corona discharge.

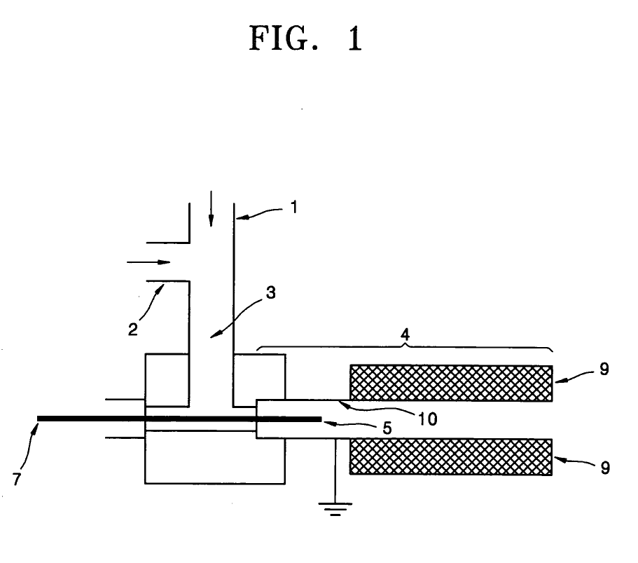

A method of producing nanoparticles by thermal decomposition using corona discharge may be realized using an apparatus for preparing nanoparticles shown in FIG. 1.

FIG. 1 is a schematic view of an apparatus for preparing nanoparticles according to an embodiment of the present invention.

The apparatus for preparing nanoparticles of FIG. 1 will now be described in detail, and the method of producing nanoparticles by thermal decomposition using corona decomposition according to an embodiment of the present invention will now be described with reference to FIG. 1.

Referring to FIG. 1, the apparatus for preparing nanoparticles includes a precursor inlet 1, a carrier gas inlet 2, a mixing unit 3 for mixing a precursor and a carrier gas, a thermal decomposition reactor 4, and a corona discharge portion 5.

Although the precursor inlet 1 and the carrier gas inlet 2 are separated in FIG. 1, the precursor inlet 1 and the carrier gas inlet 2 can be integrated into a single inlet.

The mixing unit 3 may be located adjacent to the precursor inlet 1 and the carrier gas inlet 2, and can be equipped with any mixing device for conventional thermal decomposition to mix the precursor and the carrier gas.

The thermal decomposition reactor 4 is located adjacent to the mixing unit 3. The precursor transferred from the mixing unit 3 are thermally decomposed and aggregated in the thermal decomposition reactor 4.

The thermal decomposition reactor 4 includes the corona discharge portion 5, which is connected to a corona discharge voltage supplier 7. The corona discharge portion 5 may be located before a thermal source 9 of the thermal decomposition reactor 4. The corona voltage supplier 7 supplies a voltage to the corona discharge portion 5 to cause corona discharge, and thus, products obtained by decomposing the precursor or initial aggregated products thereof, which is used to produce nanoparticles, have the same polarities.

The thermal decomposition reactor 4 includes the thermal source 9 that can maintain the temperature of the thermal decomposition reactor 4 at 850□ to 1150□. Uncharged neutral precursors are aggregated into the electrically charged thermally decomposed product or initial aggregated product thereof, which is electrically charged, in the vicinity of the corona discharge portion 5 of the thermal decomposition reactor 4. In this case, the thermally decomposed products or initial aggregated products thereof, which is the center of the aggregation, does not aggregate each other due to a repulsive force because they have the same polarities, thus suppressing the growth of nanoparticles. Accordingly, the production and sizes of nanoparticles can be controlled simultaneously. That is, the apparatus shown in FIG. 1 can be used to produce fine nanoparticles whose sizes have low distribution.

As a result, when the apparatus for producing nanoparticles shown in FIG. 1 according to the present embodiment is used, a subsequent separate process, such as a process for narrowing the size distribution of produced nanoparticles, is not required. That is, fine nanoparticles with uniform sizes can be produced without a subsequent process.

A method of producing nanoparticles by thermal decomposition using corona discharge according to an embodiment of the present invention will now be described in detail with reference to FIG. 1.

The method of producing nanoparticles using the apparatus shown in FIG. 1 includes: providing a precursor and a carrier gas through the precursor inlet 1 and the carrier gas inlet 2, respectively; mixing the provided precursor and carrier gas in the mixing unit 3; providing the mixture of the precursor and the carrier gas to the thermal reactor 4 to thermally decompose the precursor; electrically charging thermally decomposed products of the precursor or initial aggregated products thereof which are formed almost at the same time as the thermal decomposition of the precursor through corona discharge in the corona discharge portion 5 such that thermally decomposed products of the precursor or initial aggregated products thereof have the same polarities; and allowing the charged thermally decomposed products or initial aggregated products to be aggregated with neutral thermally decomposed products, thus growing nanoparticles.

The precursor and the carrier gas for nanoparticles can be provided through separate inlets or a single inlet. The provided precursor and carrier gas are sufficiently mixed in the mixing unit 3. The mixture of the precursor and the carrier gas is transferred to the thermal decomposition reactor 4 which is disposed adjacent to the mixing unit 3.

When the precursor is transferred to the thermal decomposition reactor 4, thermal decomposition starts. At this time, the thermally decomposed products of the precursor have the same polarities due to the corona discharge occurring at the corona discharge portion 5. When the precursor is thermally decomposed, the result is aggregated to form an initial aggregated product, which is used to grow a nanoparticle. These Initial aggregated products have the same polarities due to corona discharge occurring at the corona discharge portion 5. The thermally decomposed products of the precursor and the initial aggregated products thereof are aggregated in the thermal decomposition reactor 4. In this case, however, the aggregation does occur between the charged thermally decomposed products or the charged initial aggregated products thereof and neutral thermally decomposed products, because charged thermally decomposed products or charged initial decomposition products have the same polarities so that they are separated due to a repulsive force. Nanoparticles can grow due to the aggregation, but a further growth of nanoparticles is suppressed because identically charged thermally decomposed products or initial aggregated products thereof cannot be aggregated together. Accordingly, the size of the nanoparticles can be controlled when the nanoparticles are produced. That is, by applying the same electric charge to each of the decomposition products of the precursor with corona discharge, fine nanoparticles with a narrow size distribution can be produced. As a result, an additional process for classifying nanoparticles according to size is not required.

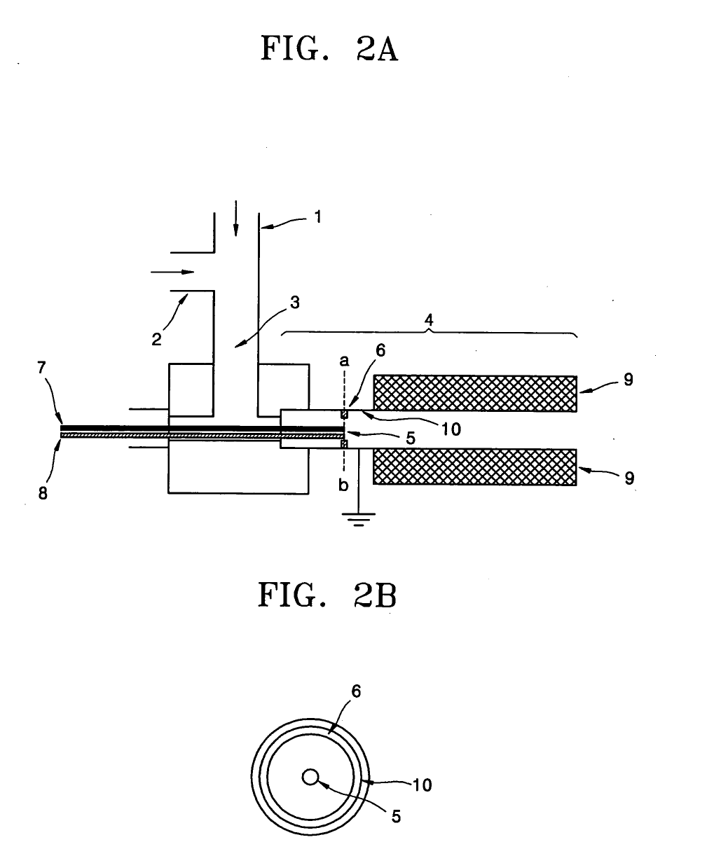

According to a method of producing nanoparticles according to another embodiment of the present invention, in operation of electrically charging, a ring that contacts the thermal decomposition reactor and surrounds a corona discharge portion may be formed. A voltage which is lower than the voltage supplied for corona discharge is supplied to the ring, thus generating an electric field between the center of the corona discharge and the ring. Thermally decomposed products or initial aggregated products thereof pass through the electric field to obtain the same polarities. The present embodiment can be realized by using an apparatus for forming nanoparticles shown in FIG. 2A.

FIG. 2A is a schematic view of an apparatus for producing nanoparticles according to another embodiment of the present invention. The apparatus for producing nanoparticles shown in FIG. 2A will now be described, and a method of producing nanoparticles in which a ring, which contacts the thermal decomposition reactor and surrounds the corona discharge portion, is introduced will be described in detail with reference to FIG. 2A.

The apparatus for producing nanoparticles shown in FIG.2A is different from the apparatus for producing nanoparticles shown in FIG. 1 in that a ring 6 which contacts an inner wall 10 of the thermal decomposition reactor 4, is connected to a ring voltage supplier 8 and encompasses the corona discharge portion 5 is formed.

Although the ring 6 is illustrated as small spots in FIG. 2A, the ring 6, in fact, contacts the inner wall 10 of the thermal decomposition reactor 4. In order to clarify the ring 6, a vertical sectional view taken along line a-b shown in FIG. 2A is illustrated in FIG. 2B. Referring to FIG. 2B, the ring 6 contacts the inner wall 10 of the thermal decomposition reactor 4, and the corona discharge portion 5 is located inside the ring 6. The corona discharge portion 5 may be located at the center of the ring 6. In addition, the ring 6 may be perpendicular to a direction in which a material flows in the thermal decomposition reactor 4, thus increasing the efficiency of the electric field. However, the location of the ring 6 is not limited thereto.

The ring voltage supplier 8 that supplies a voltage to the ring 6 may be supplied with a voltage of 0 kV to −10 kV, preferably, 0 kV to −4 k.

A connecting member between the ring voltage supplier 8 and the ring 6, and the ring 6 may be composed of any conductive material to which a voltage can be applied, such as a tungsten wire.

The ring 6 is provided with a voltage that is lower than the voltage supplied for corona discharge, thereby generating an electric field surrounding the corona discharge. When the ring 6 is not formed, corona discharge occurring at the corona discharge portion 5 results in an electric potential between the corona discharge portion 5 and the inner wall 10 of the thermal decomposition reactor 4, thereby forming an electric field between the inner wall 10 of the thermal decomposition reactor 4 and the corona discharge portion 5. Thermally decomposed products or initial aggregated products thereof which are electrically charged due to corona discharge in the corona discharge portion 5 move and are deposited on the inner wall 10 of the thermal decomposition reactor 4 because the electric potential is low at the inner wall 10 of the thermal decomposition reactor 4. As a result, the density of produced nanoparticles can be decreased. Such a decrease of the density can be prevented by installing the ring 6.

A voltage supplied to the ring voltage supplier 8 is lower than the voltage supplied to the corona discharge portion 5 so that the ring 6 has a lower voltage than the corona discharge portion 5 and thus an electric field is formed between the corona discharge portion 5 and the ring 6. As a result, an electric field does not extend to the inner wall 10 of the thermal decomposition reactor 4 and thermally decomposed products or initial aggregated products thereof, which have the same polarities, are not attached to the inner wall 10 of the thermal decomposition reactor 4.

Accordingly, nanoparticles produced using the apparatus for forming nanoparticles according to another embodiment of the present invention shown in FIG. 2A may have uniform sizes and high density.

A method of producing nanoparticles using the apparatus which further includes a ring contacting the thermal decomposition reactor and encompassing corona discharge portion, will be described in detail with reference to FIG. 2A.

The method of producing nanoparticles according to the present embodiment of the present invention includes: providing a precursor and a carrier gas through the precursor inlet 1 and the carrier gas inlet 2, respectively; mixing the provided precursor and carrier gas in the mixing unit 3; providing the mixture of the precursor and the carrier gas to the thermal decomposition reactor 4 to thermally decompose the precursor; passing thermally decomposed products or initial aggregated products thereof through an electric field formed between the corona discharge portion 5 and a ring 6 and encompassing the corona discharge portion 5 by supplying a voltage to the ring 6 that is lower than the voltage provided for corona discharge so that thermally decomposed products or initial aggregated products have the same polarities; and allowing the charged thermally decomposed products or initial aggregated product which have the same polarities to be aggregated with neutral thermally decomposed products, thus growing nanoparticles.

That is, in the present method of producing nanoparticles, the ring 6 contacting the inner wall 10 of the thermal decomposition reactor 4 is supplied with a voltage that is lower than the voltage supplied to the corona discharge portion 5, thereby generating an electric field between the corona discharge portion 5 and the ring 6. Thermally decomposed products or initial aggregated products thereof pass through the electric field to obtain the same polarities.

The voltage supplied to ring 6 may be in the range of 0 kV to −10 kV, preferably, 0 kV to −4 kV. When the supplied voltage is outside this range, that is, when the voltage difference between the corona discharge portion 5 and the ring 6 is decreased, a smaller current (ions) is generated by discharge voltage, thereby decreasing the discharge effect.

The ring 6, and a connection member between the ring 6 and the ring voltage supplier 8 may be composed of a tungsten wire, but the ring 6 and the connection member are not limited thereto.

In operation of providing voltage, the ring 6 that contacts the inner wall 10 of the thermal decomposition reactor 4 is provided with a voltage that is lower than the voltage provided for corona discharge, the formation of an electric field at the inner wall 10 of the thermal decomposition reactor 4 is prevented so that the charged thermally decomposed products and aggregated products thereof are not deposited on the inner wall 10 of the thermal decomposition reactor 4.

When the ring 6 is not installed in the thermal decomposition reactor 4 where corona discharge occurs, an electric potential difference is generated between the corona discharge portion 5 and the inner wall 10 of the thermal decomposition reactor 4, resulting in electric field between them. The thermally decomposed products or aggregated products thereof that are electrically charged due to corona discharge in the corona discharge portion 5 move to and are deposited on the inner wall 10 of the reactor 4 because the inner wall 10 of the thermal decomposition reactor 4 has a lower electric potential than the corona discharge portions. In this case, the density of the produced nanoparticles can be decreased.

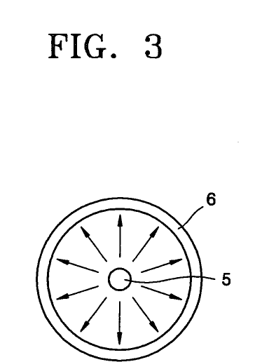

On the other hand, when the ring 6 is formed and the voltage lower than the voltage provided to the corona discharge portion 5 is supplied to the ring 6 according to the method of producing nanoparticles according to the present embodiment, the electric field is formed between the corona discharge portion 5 and the ring 6, but does not extend to the inner wall 10 of the thermal decomposition reactor 4. The direction of the electric field formed between the corona discharge 5 and the ring 6 is illustrated in FIG. 3. Referring to FIG. 3, the electric field extends radially from the corona discharge portion 5 to the ring 6, which is supplied with a lower voltage than the corona discharge.

Thermally decomposed precursors in the thermal decomposition reactor 4 are charged with the same polarities when they pass between the corona discharge portion 5 and the ring 6, and the electrically charged thermally decomposed products or initial aggregated products thereof is not deposited on the inner wall 10 of the thermal decomposition reactor 4 because the electric field is not formed at the inner wall 10 of the thermal decomposition reactor 4. Meanwhile, the electrically charged thermally decomposed products or aggregated products thereof can be deposited on the ring 6 instead of the inner wall 10 of the thermal decomposition reactor 4, but the quantity of the attached products is relatively low because the surface area of the ring 6 is much less than that of the inner wall 10 of the thermal decomposition reactor 4.

The ring 6 may contact the inner wall 10 of the thermal decomposition reactor 4. When a space is formed between the ring 6 and the inner wall 10 of the thermal decomposition reactor 4, the thermally decomposed products or initial aggregated products thereof can pass through the space where an electric field is not generated. In this case, the ratio of products that are electrically charged to products that are not electrically charged is decreased. As a result, it is difficult to obtain nanoparticles with a narrow size distribution.

The present invention will be described in further detail with reference to the following examples. These examples unit are illustrative purposes only and are not intended to limit the scope of the present invention.

### EXAMPLE 1

A tungsten wire for corona discharge was placed in a thermal decomposition reactor of an apparatus for producing nanoparticles by thermal decomposition manufactured according to the method disclosed in Korea Patent No. 2004-0070818.

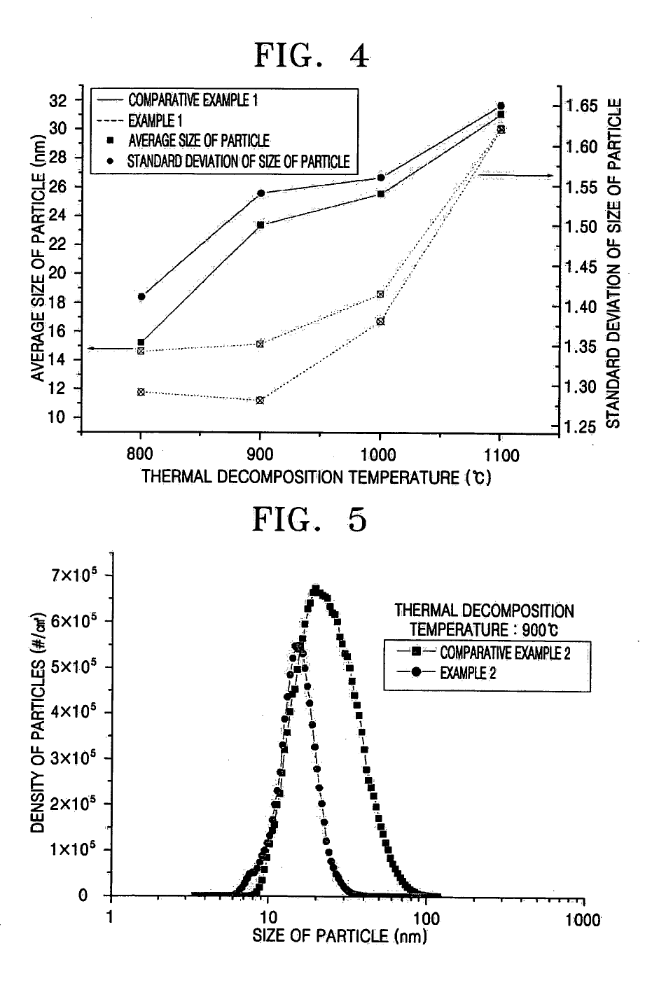

Silane (SiH₄) was provided to the apparatus at 30 sccm, and N₂ as a carrier gas was provided to the apparatus at 3000 sccm. Then, corona discharge occurred at −8 kV and the temperature for thermal decomposition was maintained in the range of 800 to 1100□ to produce silicon nanoparticles. The corona discharge was performed by supplying a voltage of −8 kV to an outside end of the tungsten wire. The average size of the nanoparticles and the standard deviation of sizes were measured, and the results are shown in FIG. 4.

### COMPARATIVE EXAMPLE 1

Nanoparticles were produced in the same manner as in Example 1 except that the corona discharge was not used. The average size of the nanoparticles and the standard deviation of sizes were measured and the results are shown in FIG. 4 in which the results of Example 1 are also shown.

### EXAMPLE 2

Nanoparticles were produced in the same manner as in Example 1 except that the temperature for thermal decomposition was 900□. The density with respect to the size of nanoparticles was measured and the results are shown in FIG. 5□

### COMPARATIVE EXAMPLE 2

Nanoparticles were produced in the same manner as in Example 2 except that the corona discharge was not used. The density with respect to the size of nanoparticles was measured and the results are shown in FIG. 5 in which the results of Example 2 are also shown.

### EXAMPLE 3

A tungsten wire for corona discharge was placed in a thermal decomposition reactor of an apparatus for producing nanoparticles by thermal decomposition manufactured according to the method disclosed in Korea Patent No. 2004-0070818.

A ring was prepared using a tungsten wire. The size of the ring was such that the ring contacted the inner wall of the thermal decomposition reactor of the apparatus. The prepared ring was placed inside of the thermal decomposition reactor of the apparatus such that the ring encompasses the corona discharge portion.

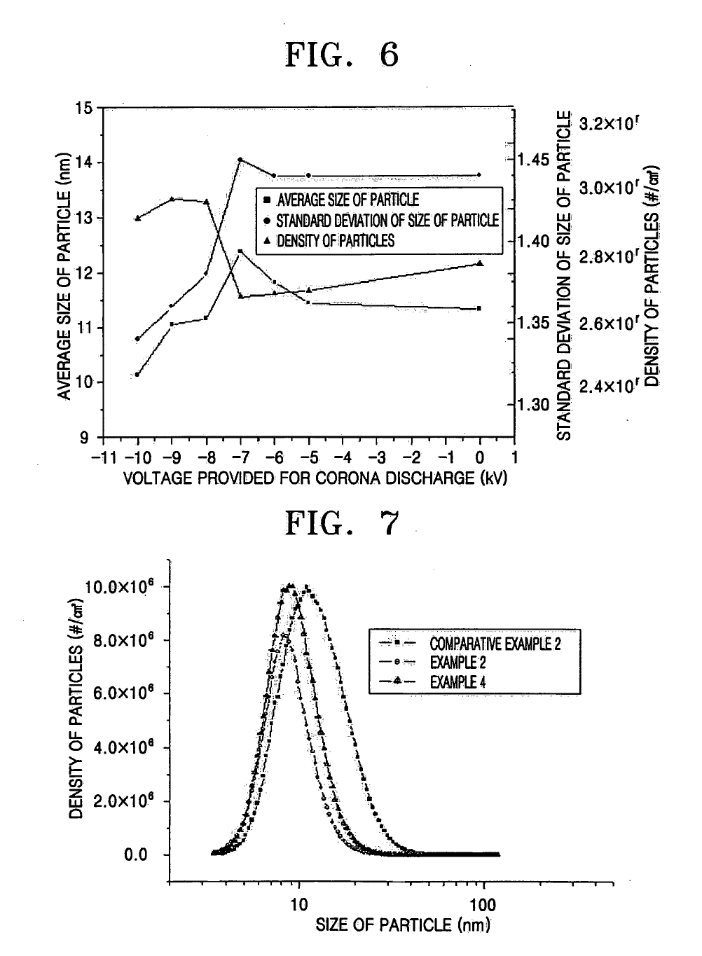

Silane (SiH₄) was provided to the apparatus at 30 sccm, and N₂ as a carrier gas was provided to the apparatus at 3000 sccm. The temperature for thermal decomposition was 900□, and corona discharge occurred at a voltage of 0 kV to −10 kV. In this case, the tungsten wire ring that contacted the thermal decomposition reactor was provided with 0 kV. The average size, density of nanoparticles produced and the standard deviation of size with respect to the voltage supplied for corona discharge were measured, and the results are shown in FIG. 6.

### EXAMPLE 4

Nanoparticles were produced in the same manner as in Example 3 except that corona discharge was carried out at only −8 kV. The density with respect to the size of nanoparticles was measured and the results are shown in FIG. 7, in which the results of Example 2 and Comparative Example 2 are also shown.

As described above, according to the present invention, nanoparticles according to Example 1 in which corona discharge occurred were smaller in size with a smaller standard deviation of size than nanoparticles according to Comparative Example 1 in which corona discharge did not occur (See FIG. 4.) Accordingly, it was confirmed that when corona discharge was added to conventional methods of producing nanoparticles using a thermal decomposition reactor, sizes of nanoparticles were controlled and size distribution was predominantly narrow.

Referring to FIG. 5, when nanoparticles were produced at a specific temperature, the size distribution of nanoparticles according to Example 2 in which corona discharge occurred was narrower than that of nanoparticles according to Comparative Example 2 in which corona discharge did not occur.

FIG. 6 illustrates the results that were obtained using nanoparticles that were produced using an apparatus for producing nanoparticles which includes a ring encompassing a corona discharge portion and a ring voltage supplier (Example 3). From FIG. 6, it was confirmed that the sizes, density, and size distribution of nanoparticles were controlled according to the voltage for corona voltage.

FIG. 7 is a graph of density with respect to size of nanoparticles according to Comparative Example 2 in which corona discharge did not occur, Example 2 in which corona discharge occurred, and Example 4, in which corona discharge occurred and the ring supplied with a voltage was installed. Referring to FIG. 7, the size distribution of nanoparticles according to Comparative Example 2 was much wider than that of nanoparticles according to Example 2. The density of nanoparticles according to Example 2 is lower than that of nanoparticles according to Comparative Example 2. However, it is just a decrease in nanoparticles with undesired sizes (see FIG. 7).

Referring to FIG. 7, it was confirmed that the density of nanoparticles with sizes suitable for commercial applications was higher in Example 4, in which a ring to which a voltage is supplied was installed, than in Example 2. That is, nanoparticles with size distributions suitable for commercial applications could be manufactured with high density by using a method of producing nanoparticles using a thermal decomposition reactor, corona discharge, and a ring to which a voltage is provided.

According to an apparatus and method for producing nanoparticles according to the present invention, nanoparticles having a desired size distribution can be manufactured. In addition, the use of a ring can increase the density of nanoparticles with a narrow size distribution.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of producing nanoparticles using gas-to-particle conversion, wherein gas discharge is induced in products obtained by decomposing a precursor or initial aggregated products of the decomposed products so that the decomposed products of the precursor or the initial aggregated products thereof are electrically charged with same polarities, thus forming nanoparticles with a narrow size distribution.

2. The method of claim 1, wherein the gas-to-particle conversion is performed by laser ablation, thermal decomposition, high-frequency sputtering, or plasma processing.

3. The method of claim 1, comprising:
providing a precursor of a nanoparticle and a carrier gas to a thermal decomposition reactor to thermally decompose the precursor;
electrically charging the thermally decomposed products of the precursor or the initial aggregated products thereof in the thermal decomposition reactor by corona discharge so that the thermally decomposed products of the precursor or the initial aggregated products thereof have the same polarities; and
growing nanoparticles by allowing the thermally decomposed products or the initial aggregated products to be aggregated with the same polarities with neutral thermally decomposed products.

4. The method of claim 3, wherein the operation of electrically charging further comprises:
providing a voltage that is lower than a voltage provided for corona discharge to a ring that contacts the thermal decomposition reactor and surrounds a portion where the corona discharge occurs, thus generating an electric field between the center of the corona discharge and the ring so that thermally decomposed products of the precursor or initial aggregated products thereof have the same polarities.

5. The method of claim 3, wherein the corona discharge occurs at a voltage of −4 kV to −20 kV.

6. The method of claim 4, wherein the ring is provided with a voltage of 0 kV to −10 kV.

7. The method of claim 1, wherein the nanopaticle is a nanoparticle of metal, an alloy, a ceramic, a semiconductor, or a composite compound.

8. An apparatus for producing nanoparticles, the apparatus comprising:
a precursor inlet and a carrier gas inlet;
a thermal decomposition reactor in which the precursor and the carrier gas are introduced and the precursor is thermally decomposed and aggregated; and
a corona discharge portion for corona discharge that is located in the thermal decomposition reactor and connected to a corona discharge voltage supplier.

9. The apparatus of claim 8, further comprising a ring which contacts the thermal decomposition reactor and is connected to a ring voltage supplier and encompasses the corona discharge portion, wherein the ring is supplied with a voltage that is lower than a voltage supplied for corona discharge to generate an electric field between the corona discharge portion and the ring.

10. The apparatus of claim 8, wherein the corona discharge voltage supplier is provided with a voltage of −4 kV to −20 kV.

11. The apparatus of claim 9, wherein the ring voltage supplier is provided with a voltage of 0 kV to −10 kV.

12. The apparatus of claims 11, wherein the nanoparticle is a nanoparticle of metal, an alloy, a ceramic, a semiconductor, or a composite compound.

13. The method of claim 4, wherein the corona discharge occurs at a voltage of −4 kV to −20 kV.
